(19) United States
(12) Patent Application Publication
(10) Pub. No.: US 2015/0216498 A1
(43) Pub. Date: Aug. 6, 2015

(54) Geometric Characterization and Calibration of a Cone-Beam Computer Tomography Apparatus

(71) Applicant: ORANGEDENTAL GMBH & CO. KG, Biberach (DE)

(72) Inventors: Ralf Kurt Willy Schulze, Rosenheim (DE); Daniel GROß, Mainz (DE); Ulrich-Alexander Heil, Mainz (DE); Elmar Schömer, Saulheim (DE); Ulrich Schwanecke, Wiesbaden (DE)

(21) Appl. No.: 14/422,492

(22) PCT Filed: Aug. 20, 2013
(86) PCT No.: PCT/EP2013/067266

§ 371 Date: Feb. 19, 2015

Related U.S. Application Data
(60) Provisional application No. 61/691,043, filed on Aug. 20, 2012.
(60) Provisional application No. 61/692,320, filed on Aug. 23, 2012.

(51) Int. Cl. A61B 6/00

(52) U.S. Cl. CPC A61B 6/583; A61B 6/4085

(57) ABSTRACT

It is described a method for determining values of geometry parameters of a cone beam computer tomography apparatus, the method comprising: (a) obtaining x-ray projection data captured by the detector from at least three calibration objects arranged at mutually different positions, the x-ray projection data comprising for each calibration object plural projections at different rotation angles; (b) determining for each calibration object a respective ellipse representation from the respective plural projections; (c) performing a random search across candidate values of the geometry parameters for determining the values of the geometry parameters, wherein a cost function depending on the ellipse representations and the geometry parameters is optimized.

### FIELD OF THE INVENTION

The present invention relates to a method and to an arrangement for determining values of geometry parameters of a cone-beam computer tomography apparatus as well as to a program element and a computer readable medium which are adapted to control or carry out such a method and further relates to a cone-beam computer tomography apparatus.

### ART BACKGROUND

Flat-panel cone-beam CTs (CBCTs) are widely used in medicine for three-dimensional reconstructions, but also have applications in industry and science. The data basis is formed by a large number of X-ray projection images which are uniformly distributed around the object of interest. In most cases there is a rotating C-arm composed of an X-ray tube and a flat rectangular detector. To permit a volumetric reconstruction the precise knowledge of the geometric alignment of the detector and the X-ray tube in relation to the rotational axis is an indispensable precondition. Otherwise various artifacts can be observed.

There are numerous methods present in literature for calibrating a CBCT with different restrictions on the device geometry or the calibration phantom which may also be referred to as calibration object. Only some take out-of-plane rotations into account. Common to all of these methods is the need for a priori information about the used calibration phantom. Their direct method, however, requires the detector to be oriented parallel to the rotation axis and hence only accommodates one of two out-of-plane rotations (here denoted by σ, θ). While such methods usually allow someone to calibrate each projection separately, the calibration accuracy is additionally limited by knowledge of the precise adjustment of the point-like markers. Other methods take more sophisticated phantoms into account. The method of Yang et al.¹ also uses a phantom consisting of arbitrarily positioned markers, the relative positions of which may be unknown. Yang et al. only need a rough measurement of the distance between two of the markers to adjust the focus-to-object distance, but they assumed out-of-plane rotations to be negligible.

There may be a need for a method and an arrangement for determining values of geometry parameters of a cone-beam computer tomography apparatus, for a program element, for a computer readable medium and for a cone-beam computer tomography apparatus, wherein the relative arrangement and orientation of components of the cone-beam computer tomography apparatus can be determined in a simple and reliable manner with high accuracy.

This need may be met by the subject-matter of the independent claims. The dependent claims specified particular embodiments for carrying out the invention.

### SUMMARY OF THE INVENTION

According to embodiments of the present invention, there are provided a method and an arrangement for determining values of geometry parameters of a cone-beam computer tomography apparatus, a program element, a computer readable medium and a cone-beam computer tomography apparatus, wherein the relative arrangement and orientation of components of the cone-beam computer tomography apparatus can be determined in a simple and reliable manner with high accuracy.

According to an embodiments of the present invention it is provided a method for determining values of geometry parameters of a cone beam computer tomography apparatus, the geometry parameters in particular specifying an arrangement of a two-dimensional x-ray detector, an arrangement of a rotation axis of a relative rotation between an object and a mechanically coupled x-ray source/detector system, and an arrangement of a focus of the x-ray source, the method comprising (a) obtaining x-ray projection data captured by the detector from at least three calibration objects arranged at mutually different positions, the x-ray projection data comprising for each calibration object plural projections at different rotation angles; (b) determining for each calibration object a respective ellipse representation from the respective plural projections; (c) performing a random search across candidate values of the geometry parameters for determining the values of the geometry parameters, wherein a cost function depending on the ellipse representations and the geometry parameters (or the candidate values thereof) is optimized.

The geometry parameters may in particular define or specify a relative arrangement/relative orientation of the 2-dimensional x-ray detector, the rotation axis and the x-ray source. The correct values of the geometry parameters may be required in order to reconstruct a volume density of an examination object which has been measured using the cone-beam computer tomography apparatus by measuring plural projections of the examination object obtained under different viewing directions, i. e. acquired at different rotation angles of a relative rotation between the examination object and the mechanically coupled x-ray source/detector system.

The cone-beam computer tomography apparatus may be of any type and may be adapted to for example examine biological objects, such as a human being or an animal, or may be adapted to examine industrial articles.

The relative rotation between the object and the mechanically coupled x-ray source/detector system may be assumed to be an exactly circular rotation. Thereby, the method may be simplified, while it can be shown that this is a realistic assumption.

Obtaining the x-ray projection data may comprise supplying or acquiring or reading electrical and/or optical signals which are indicative of the x-ray projection data. Additionally, obtaining the x-ray data may comprise acquiring measurement data using the cone-beam computer tomography apparatus from projections of the at least three calibration objects and afterwards providing the measurement data in a computer readable form as electrical and/or optical signals.

In particular, the x-ray projection data may comprise plural images, each image carrying data which are indicative of one or more projection of one or more calibration object. In particular, the plural projections at different rotation angles of a first calibration object may be collected in a single first image and the plural projections at different rotation angles of a second calibration object may be connected in a second image. Further, the plural projections of any of the other of the at least three calibration objects may be connected in a further image. In particular, the first image, the second image and the at least one further image may then be successively supplied from which the x-ray projection data may be assembled. Alternatively, the first image, the second image and the at least one further image may be assembled into a total image harboring the information about all projections of all the calibration objects, wherein the projections have been obtained under different rotation angles of the respective calibration object.

In particular, the at least three calibration objects may be simultaneously projected onto the x-ray detector upon irradiation with x-rays generated by the x-ray source at a first rotation angle. At this first rotation angle the at least three calibration objects may be fixed (in particular stand still, are stationary) relative to the coupled x-ray source/detector system and the x-ray detector may be exposed to the x-rays for a particular exposure time, while the calibration objects are stationary. Further on, the calibration objects may as a whole be rotated to a second rotation angle and the x-ray detector may be exposed during an exposure time to the x-rays, while the calibration objects are again stationary (i.e. fixed relative to the x-ray source/detector system). This procedure may be carried out for further rotation angles being different from the first rotation angle and the second rotation angle.

Thereby, the x-ray projection data may be collected in a relatively fast and simple manner, in particular not requiring any alignment of individual images taken from individual calibration object or taken for individual rotation angles.

The x-ray projection data may comprise positionally resolved intensity data across an x-ray sensitive area of the detector, wherein the intensity distribution (in particular comprising intensity peaks) across this x-ray sensitive area of the detector is due to the calibration objects, in particular their absorption of the x-ray radiation. In particular, the calibration objects may be manufactured from material which absorbs the x-rays generated by the x-ray boo source, such that only 0% to 50%, in particular 0% to 20% of the intensity of the impinching x-rays is transmitted through the respective calibration object. Thereby, a intensity peaks may be generated and a high contrast of the intensity distribution may be obtained, thereby simplifying the method for determining the values of the geometry parameters.

The x-ray source may be adapted to generate x-rays such that the x-rays originate from a focus of the x-ray source in a star-like manner (e.g. radially propagating in all directions from the focus). The focus of the x-ray source may for example be an impinchment point or impinchment area (at an anode material) of an impinchment of electrons which have been accelerated to a sufficient energy, in order to excite photons in the x-ray wavelength range. Upon impinchment of the electrons at a target point of the anode material (which may be more positively charged relative to a cathode from which the electrons originate) the impinching electrons may excite electrons of the anode material into higher energy levels, wherein these excited electron may then fall back to lower energy levels whereby the photons in the x-ray wavelengths range may be emitted. Thereby, x-rays may be generated which originate from the focus of the x-ray source and propagate in different directions from the focus in a star-like or radial manner. Thereby, large examination objects or a large examination volume may be examined using the cone-beam computer tomography apparatus.

Determining for each calibration object the respective ellipse representation from the respective plural projections (originating from the same calibration object) may comprise extracting intensity peaks from the x-ray projection data which are due to a same calibration object projected under different rotation angles. Further, centers of the intensity peaks may be determined. Still further, the plural centers corresponding to the plural different rotation angles may be positionally characterized or determined and from the positions of the centers an ellipse may be fitted such as to minimize a deviation between the ellipse and the positions of the centers. Thereby, any fitting routine may be applied, as is known in the art.

Performing the random search may comprise to explore different possible values of the geometry parameters and assessing a quality of the possible values using the cost function. Thereby, the cost function may measure or allow to derive a degree of matching between the (previously obtained, in particular from measurements and computations) ellipse representations and a theoretically calculated image which depends on the currently explored candidate values of the geometry parameters.

The method may ensure an accurate determination of the values of the geometry parameters in a simple and reliable manner. In particular, all geometry parameters relevant for reconstruction of an examination object using projections of the examination object may be determined using the method, without having any prior knowledge on any particular value of the geometry parameters. Further, no prior knowledge is necessary regarding the relative arrangement of the calibration objects. Thereby, manufacturing the calibration objects or a structure carrying all calibration objects may be simplified compared to conventional methods.

It must be emphasized that no information about the relative position of the markers is required, nor are manual measurements required, and even the real pixel size can be neglected. The problem may be formulated as a non-linear optimization problem based on the geometric restrictions described above and the ellipse parameters as input data and may be solved iteratively.

Although no metric information about the phantom/calibration objects has to be known, all parameters can be determined with high accuracy.

According to an embodiment of the present invention, the performing of the random search comprises describing each ellipse representation in the form of a virtual ellipse in a plane parallel to the rotation axis modified by a geometry related mapping defined by the geometry parameters.

The virtual ellipse may represent a projection of the respective calibration object projected under different rotation angles, when the x-rays sensitive area of the detector would be situated exactly within an ideal plane (x-y-plane), wherein this plane is parallel to the rotation axis and parallel to an axis (x-axis) which is perpendicular to the rotation axis and which is also perpendicular to an axis (z-axis) perpendicular to the rotation axis and running through the focus of the x-ray source.

In particular, the method does not assume that the x-ray sensitive area of the detector lies in this ideal plane and the method allows to determine the orientation of the real x-ray detector relative to this ideal plane. Thereby, reconstructions of examination objects may be obtained in a more accurate manner.

The virtual ellipse may be described in a simple manner, thereby simplifying the method. In particular, the virtual ellipse may be described using only two parameters, e. g. specifying the position of the respective center of the respective calibration object, in particular relative to the rotation axis (distance from it) and in a direction parallel to the rotation axis.

According to an embodiment of the present invention, the describing the ellipse representation for each calibration object comprises the relation: \tilde{C}=\tilde{G}^{T}·\tilde{C}_{c}·\tilde{G}, wherein \tilde{C} is the ellipse representation as determined from the projections of the calibration object, \tilde{C}_{c} is the virtual ellipse in the plane parallel to the rotation axis, and \tilde{G} is the geometry related mapping depending on the geometry parameters, \tilde{G}^{T} denoting the transpose of \tilde{G}, wherein \tilde{C}, \tilde{C}_{c}, and \tilde{G} are representable as 3×3 matrices.

The relation may be established by a matrix equation, wherein on the right-hand side the transprose of the matrix G is multiplied by a matrix \tilde{C}_{c} representing the virtual ellipse which is multiplied by the matrix \tilde{G}. The ellipse representation on the left-hand side may represent the ellipse as determined from the plural projections of the respective calibration object.

The ellipse representation may be defined using nine real values. Thereby, the relation representing a matrix equation may be equivalent to nine equations relating scalar quantities. In particular, the geometry parameters may be comprised in the matrix \tilde{G}. In particular, there may be six values of the geometry parameters required to be determined in order to completely characterize the configuration of the cone-beam computer tomography apparatus. These six values of the geometry parameters may be derived from a variety of relations as defined above which are available from the at least three calibration objects. Thereby, the method may be simplified.

According to an embodiment of the present invention, during the methods for determining events of the geometry parameters, the performing the random search comprises finding the values of the geometry parameters such that the relation is at least approximately satisfied for all ellipse representations of the calibration objects by minimization of the cost function.

Candidate values of the geometry parameter may be explored resulting in different geometry related mappings \tilde{G}. The above relation allows then to evaluate the right-hand side which is then compared to the left-hand side (the ellipse or ellipses as determined from the measured projections). Those values of the geometry parameters which lead to a relatively low deviation between the left-hand side of the above relation and the right-hand side of the above relation may then represent approximate values of the geometry parameter which are close to the optimal values of the geometry parameters.

According to an embodiment of the present invention the cost function comprises a sum of individual cost functions, wherein each individual cost function is associated with a respective ellipse representation of one of the calibration objects and measures a deviation between the ellipse representation and the virtual ellipse modified by the geometry related mapping, wherein the deviation is based on a sum of absolute differences of four points of the ellipse representation and the modified virtual ellipse, respectively, the four points being in particular defined as intersections of the principal axes of the ellipse with the respective ellipse.

Composing the cost function from individual cost functions being associated with the respective calibration objects may simplify the method. Further, the computation may be simplified and accelerated.

According to an embodiment of the present invention, the virtual ellipse exclusively depends on a position (h) along the rotation axis (y) of the respective calibration object and a distance (r) from the rotation axis (y) of the respective calibration object.

The position (h) along the rotation axis and the distance r from the rotation axis of the calibration objects do not need to be known in order to carry out the method. Instead, this positional information of the calibration objects is also determined during performing the method or determining the values of the geometry parameters. Thereby, it is not required to prepare or manufacture one or more calibration objects with a particular relative positioning to each other. Thereby, the method is simplified and costs for manufacturing the calibration objects may be saved.

According to an embodiment of the present invention performing the random search in the geometry parameters comprises establishing start search ranges in the geometry parameters, which are explored during the random search, wherein the start search ranges are universal for different cone-beam computer tomography apparatuses, wherein in particular for each geometry parameter a start search range by a lower bound and an upper bound is specified.

In particular, each of the geometry parameters may be associated with an individual start search range. Thereby, the start search range (of a particular geometry parameter) may be selected such as to cover all expected values for the respective values which are expected for different cone-beam computer tomography apparatuses. Further, prior knowledge, if available, may be used to restrict the size or positioning of the start search ranges. Thereby, it may be insured that during the random search the correct value of the geometry parameters is found, while the search is restricted to only those values of the geometry parameters which are theoretically/practically to be expected.

According to an embodiment of the present invention, the method comprises, after performing the random search in the geometry parameters to obtain preliminary values of the geometry parameters: performing an annealing process further minimalizing the cost function, wherein the annealing process includes establishing annealing search ranges around the preliminary values of the geometry parameters, wherein the annealing search ranges include a narrower range of values of the geometry parameters than the start search ranges.

The annealing process may further improve the preliminary values of the geometry parameter by further exploring further values of the geometry parameter which are comprised in a range around the preliminary values of the geometry parameters. Thereby, it may be enabled to more accurately determine the values of the geometry parameters.

According to an embodiment of the present invention plural annealing processes are successively performed, in which sizes of the annealing search ranges are gradually decreased after a fixed number of random searches have been performed, wherein the values of the geometry parameters are determined as a minimum of the cost function over all performed random searches using search ranges having different sizes.

In particular, the sizes of the search ranges may be decreased from one random search to a next random search in a stepwise or in a continuous manner. Thereby, it may be ensured to find an optimal value of the respective geometry parameter within the current search range.

According to an embodiment of the present invention the geometry parameters represent normalized geometry parameters from which real geometry parameters can be calculated by spatial scaling. The normalized geometry parameters may in particular comprise ratios of real geometry parameters with other geometry parameters of the computer tomography apparatus.

By determining the values of normalized geometry parameters, the method may be applied to a wide variety of different computer tomography apparatuses without changing the method. Thereby, a universal method for determining the values of the geometry parameters may be provided.

According to an embodiment of the present invention the geometry parameters include information indicative of the six quantities: (phi, sigma, psi) being three Euler angles describing the orientation of a x-ray sensitive area of the detector; ps/fdd; ox/fdd; and oy/fdd, wherein ps is the pixel size of a pixel of the x-ray sensitive area of the detector; fdd is the distance along the z-axis between a focus of the x-ray source and the detector; ox is an offset along the x-axis of an origin of the x-ray sensitive area of the detector and an x-coordinate of the focus of the x-ray source; oy is an offset along the y-axis of an origin of the x-ray sensitive area of the detector and an y-coordinate of the focus of the x-ray source, wherein the y-axis represents the rotation axis, wherein the z-axis represents an axis perpendicular to the rotation axis through the focus of the x-ray source, wherein the x-axis represents an axis perpendicular to the y-axis and to the z-axis.

Thereby, the six quantities allow to completely specify the geometric configuration or constitution of the computer tomography apparatus. Using the (correct) values of these six quantities allows to reconstruct a volume identity of an examination object from plural projections of the examination object. An absolute scaling of the reconstruction volume may be applied later on.

According to an embodiment of the present invention the area of the detector is flat and is oriented traverse to the x-y-plane, wherein phi indicates an rotation angle around the x-axis, sigma indicates an rotation angle around the z-axis, and psi indicates an rotation angle around the y-axis, to describe the orientation a x-ray sensitive area of the detector relative to an area of an ideal detector being oriented parallel to, in particular within, the x-y-plane, wherein in particular fod is the distance along the negative z-axis between the focus of the x-ray source and the rotation axis oz is the distance along the z-axis between the detector and the rotation axis such that fdd=fod+oz.

Assuming a flat x-ray sensitive area of the detector dramatically simplifies the method without introducing inacceptable inaccuracies, since the commercially available detectors usually have an x-ray sensitive area which is flat to a high position.

However, taking also into account that the area of the real detector does not lie in the x-y-plane may significantly improve a reconstruction of an examination object which has been examined using plural projections obtained by the cone-beam computer tomography apparatus. In particular, in reality, the area of the real detector may significantly deviate from the x-y-plane and disregarding this deviation may result in reconstruction artifacts, as observed in conventional systems.

According to an embodiment of the present invention each of the calibration objects comprises x-ray absorbing material distributed such as to result in a intensity distribution in a x-ray projection having a single peak such that a position of a center of absorption is derivable from the respective projection, wherein in particular each calibration object comprises a metal sphere, in particular having a diameter between 0.5 mm and 2 mm.

Configuring the calibration objects, such as to result in a single peak in a single protection at a particular rotation angle may simplify the method. Further, a metal sphere is conventionally available, thereby rendering the method cost-effective.

According to an embodiment of the present invention a relative positioning of the calibration objects is not used and/or not known to determine the values of the geometry parameters. In conventional methods often very specialized calibration structures or objects were required which were expensive and difficult to manufacture.

According to an embodiment of the present invention for each calibration object the respective plural projections are obtained at different rotating angles covering a range from 0 to 360, wherein a number of projections for each calibration object is between 50 and 200.

The plural projections may be obtained at discrete rotation angles. Restriction the number of projections may accelerate the method. Further, when the rotation angles cover a range from 0 to 360 may be more simple to fit an ellipse to the resulting intensity peaks.

According to an embodiment of the present invention each calibration object is arranged such that for all rotation angles the calibration object is projected onto the x-ray sensitive area of the detector, thus not to a region outside the detector area.

Thereby, the ellipses may be fitted in a more reliable manner, since more (in particular a maximum of) information is captured by the detector and no projection of a calibration object at a rotation angle falls outside the x-ray sensitive area of the detector.

According to an embodiment of the present invention each calibration object has a distance (r) from the rotation axis such that intensity peaks within the plural projections of a calibration object due to the projection of the calibration object have a maximal mutual distance along the x-axis which is between 70% and 100%, in particular between 85% and 100% of an extent of the detector along the x-axis, the x-axis representing an axis perpendicular to the rotation axis and perpendicular to an axis which is perpendicular to the rotation axis and runs through the focus of the x-ray source.

Thereby, the area of the detector may be effectively used for determining the values of the geometry parameters. In particular, by adapting the method such that the calibration objects are projected to outer regions of the detector while ensuring that the calibration objects are not projected to regions outside the detector may allow a more accurate determination of the values of the geometry parameters.

According to an embodiment of the present invention the calibration objects are distributed in a direction parallel to the rotation axis such that between 50% and 100%, in particular 75% to 100%, of the intensity in the x-ray projection data are captured in a first region and a second region of the x-ray sensitive area of the detector, the first region and second region each having an extent of 30%, in particular 25%, of an extent of the area of the detector in a direction parallel to the rotation axis and each including a respective outer edge of the area of the detector in the direction parallel to the rotation axis.

In particular, when the intensity peaks due to projections of the calibration objects are located in outer regions of the detector (along the rotation axis or a direction parallel to the rotation axis) ellipses are formed which have a relatively large conjugate diameter (an extent along the minor axis of the ellipse) which may allow a more accurate fitting of the ellipse representation based on the plural intensity peaks and determining the values of the geometry parameter may be performed in a more accurate manner. In particular the conjugate diameter may be sensitive to the orientation of the detector, such that a high precision of the determination of the conjugate diameter may allow an accurate determination of the orientation of the detector.

In contrast, when the intensity peaks would be located in a central region of the detector (along a direction parallel to the rotation axis), the intensity peaks would be associated with ellipses having a relatively small conjugate diameter, which may be even zero, if the respective calibration object is situated at a same position as the focus of the x-ray source (along a direction parallel to the rotation axis), in which case fitting an ellipse would be difficular if not impossible and the information about the orientation of the detector may be poor.

However when the intensity peaks due to projections of the calibration objects are located in outer regions of the detector, fitting the ellipses is facilitated and determining the values of the geometry parameter may be performed in a more accurate manner.

According to an embodiment of the present invention the calibration objects are positioned and/or oriented such that the plural projections of one of the calibration objects mutually do not overlap with the plural projections of another one of the calibration objects.

Avoiding an overlap of intensity in peaks from different calibration objects or different rotation angle may simplify the method (in particular the ellipse fitting) and improve the accuracy.

According to an embodiment of the present invention the calibration objects include between three and eight calibration objects which are distributed within a reconstruction volume, in particular located at a surface of a circular cylinder, in particular along a straight line parallel to the rotation axis.

Three to eight calibration objects may ensure an accurate determination of the values of the geometry parameter while keeping the manufacturing or structuring of the calibration structure simple and cost-effective.

According to an embodiment of the present invention determining for each calibration object a respective ellipse representation from the respective plural projections comprises at least one of the following: combining all projections of one calibration object into a single image; extracting centers of the calibration object for all projections of the calibration object; applying a border segmentation; performing an elliptical Hough transformation; applying a Kalman filter; and fitting of an ellipse to all extracted and/or processed centers.

Thereby, the ellipses may be effectively fitted from the measurement data.

According to an embodiment of the present invention it is provided a method for deriving values of geometry parameters of a cone beam computer tomography apparatus, the method comprising: performing an x-ray measurement to capture x-ray projection data of calibration objects; determining, based on the x-ray projection data of the calibration objects, the values of the geometry parameters of the cone beam computer tomography apparatus according to one of the embodiments as described above.

Performing the x-ray measurement is a technical process including generating x-rays, directly the x-rays to the calibration objects (successfully or simultaneously), absorbing a portion of the intensity of the x-rays upon transmission through the calibration objects, impinging the partial absorbed x-rays onto a x-ray sensitive area of a detector thereby detecting (positionally resolved) an intensity of the x-rays, transforming the impinging/detected x-rays into electrical/optical signals which are positionally resolved and providing the electrical/optical signals to a processor.

Further, after determining the values of the geometry parameters based on the x-ray projection data which have been obtained or measured, the values of the geometry parameters may be output at a monitor, output at a printer or maybe stored in a data storage unit, such as a semi-conductor based electronic storage, such as a flash memory, a hard disk or the like.

According to an embodiment of the present invention, it is provided a method of operating a cone beam computer tomography apparatus, the method comprising: performing a method for determining values of geometry parameters of a cone beam computer tomography apparatus or a method for deriving values of geometry parameters of a cone beam computer tomography apparatus according to an embodiment as described above; performing a further x-ray measurement to capture further x-ray projection data of an examination object different form the calibration objects; and using the values of geometry parameters to reconstruct a volume density of the examination object based on the further x-ray projection data.

Operating the cone-beam computer tomography represents a technical process involving similar steps as the method for deriving values of the geometry parameters in which, however, instead of the calibration objects, an examination object is irradiated with the x-rays under different rotation angles.

Further, different methods may be applied to construct the volume density of the examination object based on the further x-ray projection data. In particular, a back projection may be applied in a real space or a Fourier space based reconstruction methods may be applied or a combination of a real space method and a Fourier space method may be applied.

According to an embodiment of the present invention, a program element is provided, which, when being executed by a processor, is adapted to control or carry out a method of determining methods of geometry parameters or a method for dividing values of geometry parameters, is explained or described according to one of the embodiments above.

According to an embodiment of the present invention it is provided a computer readable medium, which a computer program is stored which, when being executed by a processor, is adapted to control or carry out the method of determining values of geometry parameter or a method for deriving values of geometry parameter according to one of the embodiments as described above.

It should be understood that features which have been individually or in any combination disclosed, described, explained or applied to a method for determining values of geometry parameters or to a method for deriving methods of geometry parameters may also be applied to an arrangement for determining values of geometry parameter according to a new embodiment of the presents invention and vice versa.

According to an embodiment of the present invention, it is provided an arrangement for determining values of geometry parameters of a cone beam computer tomography apparatus, the geometry parameters in particular specifying an arrangement of a two-dimensional x-ray detector, an arrangement of a rotation axis of a relative rotation between an object and a mechanically coupled x-ray source/detector system, and an arrangement of a focus of the x-ray source, the arrangement comprising: an input section adapted to obtain x-ray projection data captured by the detector from at least three calibration objects arranged at mutually different positions, the x-ray projection data comprising for each calibration object plural projections at different rotation angles; and a processor adapted to determine for each calibration object a respective ellipse representation from the respective plural projections, and to perform a random search across candidate values of the geometry parameters for determining the values of the geometry parameters, wherein a cost function depending on the ellipse representations and the candidate values is optimized.

The input section may comprise one or more terminals for receiving electrical or optical signals which are identicative for the x-ray projection data.

The input section may in particular comprise an interface for obtaining data in different formats or according to different communication protocols, such as e.g. TCP/IP, HTTP, Ethernet, file transfer protocol or the like.

The processor may comprise a semi-conductor based processor, such as a semi-conductor chip. In particular, the processor may be programmable, in particular using a program element or a computer readable medium according to an embodiment of the present invention as explained above. In particular, the processor may comprise a mathematical processor, or an arthmetic/logical unit or a graphics processor. In particular, the arrangement may be adapted to carry out a method for determining methods of geometry parameter according to an embodiment of the present invention as explained above.

According to an embodiment of the present invention, it is provided a cone beam computer tomography apparatus, comprising: a two-dimensional x-ray sensitive detector; a x-ray source adapted to generate x-rays originating from a focus and mechanically coupled to the detector; an object holder for holding an object; and an arrangement for determining values of geometry parameters of a cone beam computer tomography apparatus according to an embodiment as describe above, wherein the apparatus is adapted to allow a relative rotation between the object holder and the mechanically coupled x-ray source/detector system.

Embodiments of the present invention are now described with reference to the accompanying drawings. The present invention is not limited to the described or illustrated embodiments.

### BRIEF DESCRIPTION OF THE DRAWINGS

FIG. 1 schematically illustrates an arrangement of a focus of a x-ray source, a rotation axis and a x-ray sensitive area of a detector of a cone beam computer tomography apparatus for illustrating a method according to an embodiment of the present invention;

FIG. 2 schematically illustrates a method step during performing a method for determining values of geometry parameters according to the embodiment of the present invention;

FIG. 3(a) schematically illustrates defects of having a detector area oriented to deviate from an ideal plane;

FIG. 3(b) illustrates geometrical relationships which are consistent according to embodiments of the present invention;

FIG. 4(a) illustrates an arrangement of calibration objects according to an embodiment of the present invention;

FIG. 4(b) schematically illustrates x-ray projection data derived from calibration objects as illustrated in FIG. 4(a) and utilized in a method for determining values of geometry parameter according to the embodiment of the present invention; and

FIG. 4(c) schematically illustrates other x-ray projection data originating from projecting other calibration objects or selecting portions of the data in FIG. 4(b) according to an embodiment of the present invention.

### DETAILED DESCRIPTION OF EMBODIMENTS

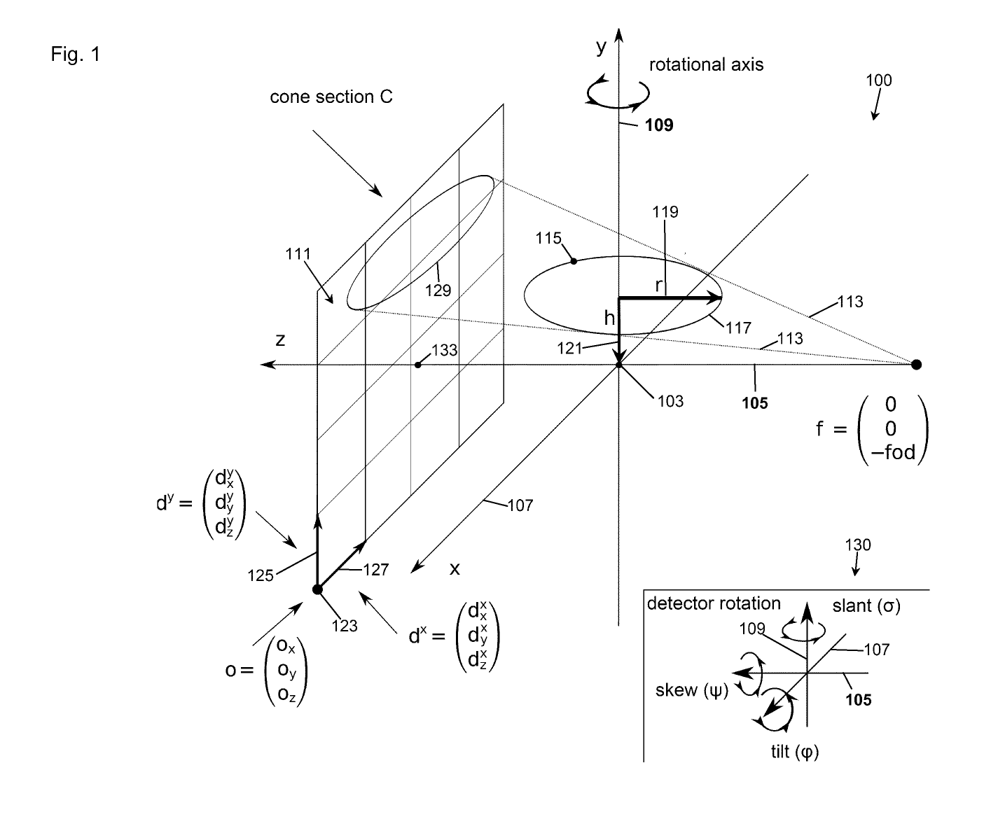

FIG. 1 schematically illustrates a geometric configuration of a cone beam computer tomography apparatus for which the values of geometry parameters are determined according to the embodiment of the present invention.

The point 101 in the scheme 100 illustrated in FIG. 1 indicates or represents a focus of an otherwise not illustrated x-ray source of the computertomography apparatus. The focus 101 is arranged at a distance fod away from an origin 103 of a Cartesian coordinative System, wherein the focus 101 is arranged on the z-axis 105. The coordinative system further comprises an x-axis 107 and a y-axis 109 to define an orthogonal coordinative system.

Mechanically coupled to the focus 101 of the x-ray source is a detector comprising a x-ray sensitive area 111 which comprises x-ray sensitive pixel elements for positionally resolving and detecting intensity values of x-rays from which exemplarily x-rays 113 are illustrated in FIG. 1. In fact other x-rays originate from the focus 101 and propagate in different directions in a star-like manner.

An examination object (not shown) or a calibration object (sphere 115) arranged between the mechanically coupled system of the focus 101 of the x-ray source and the detector having the area 111 may be rotated around the rotational axis 109 which in the illustration is along the y-axis. Exemplarily, in FIG. 1 a calibration object 115 is indicated which upon rotation about the rotational axis 109 describes a so called y-orbit 117 which lies in a plane perpendicular to the rotational axis 109. In particular, the orbit 117 is a circular orbit. The calibration object 115 is arranged a distance 119 (also referred to as r) away from the rotation axis 109 and is arranged a height 121 (also referred to as h) above the origin 103 of the coordinate system defined by the axis 105, 107 and 109. The area 111 of the detector has an origin 123 which is given by the vector (ox,oy,oz) in the coordinate system illustrated in FIG. 1. Further, the detector area 111 lies within a plane defined by the vectors 125 and 127 which are illustrated in FIG. 1.

By irradiating the calibration object 115 using the x-ray source having the focus 101, intensity peak are detected by the x-ray sensitive area 111 which are arranged on an elliptical curve 129. In particular, the elliptical curve 129 may be an ellipse representation associated with the calibration object 115, wherein this ellipse orientation is determined according to an embodiment of the present invention, in order to derive values of geometry parameters. The geometry parameters may for example define the positioning of the focus 101, the positioning of the origin 123 of the area 111 of the detector and the orientation of the plane of the area 111 which is defined by the vectors 125 and 127. In particular, the vectors 125, 127 may be described by a detector rotation as is scetched in the insert 130 in FIG. 1, wherein the detector orientation is described relative to the x-y-plane (also called the ideal plane) and rotations around the x-axis, the y-axis and z-axis by respective angles as is apparent from the insert 130 of FIG. 1.

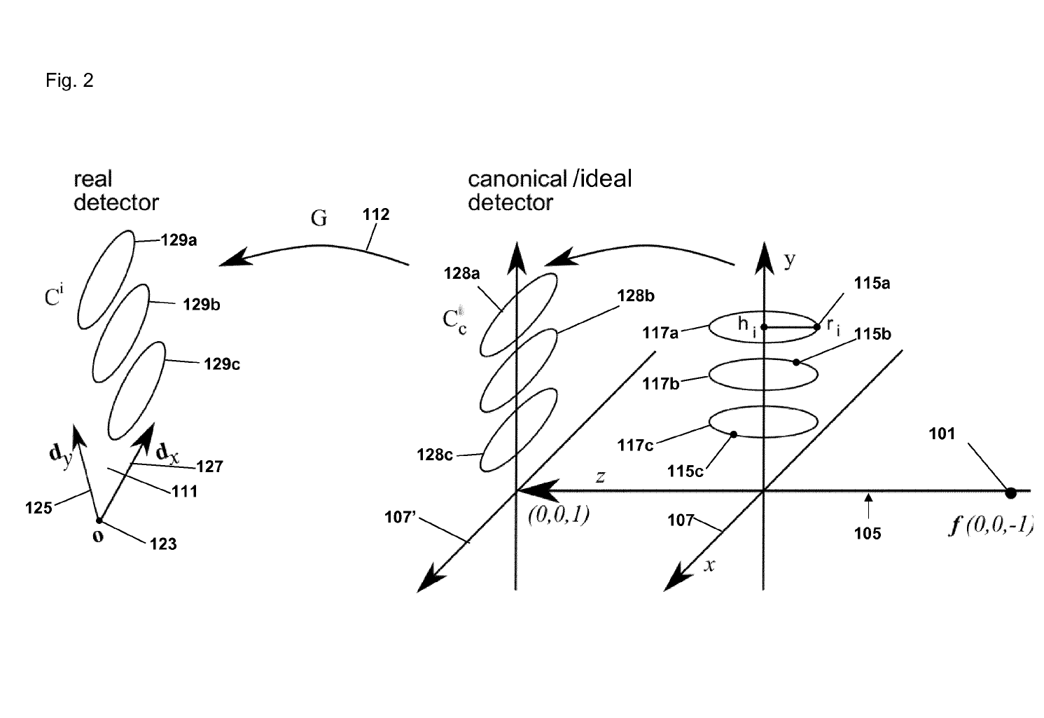

FIG. 2 schematically illustrates a stepwise mapping of projections of the calibration objects 115a, 115b and 115c onto the x-y plane, representing a plane of an area of an ideal detector, and from there to the area 111 of the real detector, wherein the orientation of the area 111 is given by the vectors dy (125) and dx (127).

The calibration objects 115a, 115b and 115c describe orbits 117a, 117b and 117c respectively, when rotated around the rotation axis 109. In the x-y plane or a plane parallel to the x-y plane (i.e. shifted by a distance z) the calibration objects 115a, 115b and 115c are projected into virtual ellipses 128a, 128b and 128c, respectively. These virtual ellipses are also referred to as Cc. Using a geometry related mapping denoted as G (112) in FIG. 2 the virtual ellipse 128a, 128b and 128c are mapped to the ellipse representations 129a, 129b and 129c which are detected by the detector having the x-ray sensitive area 111.

While the projection 110 from the calibration objects to the (ideal) plane parallel to the x-y plane does not depend on the orientation and positioning of the x-ray sensitive area 111 of the detector, the mapping 112 described by the geometry related mapping G depends on the orientation and positioning of the area 111 of the detector, thus on the origin 123, and the vectors 125 and 127. The decomposition of the complete projection from the calibration objects to the real detector into the operations 110 and 112 allowes a simple method for determining the values of the geometry parameters.

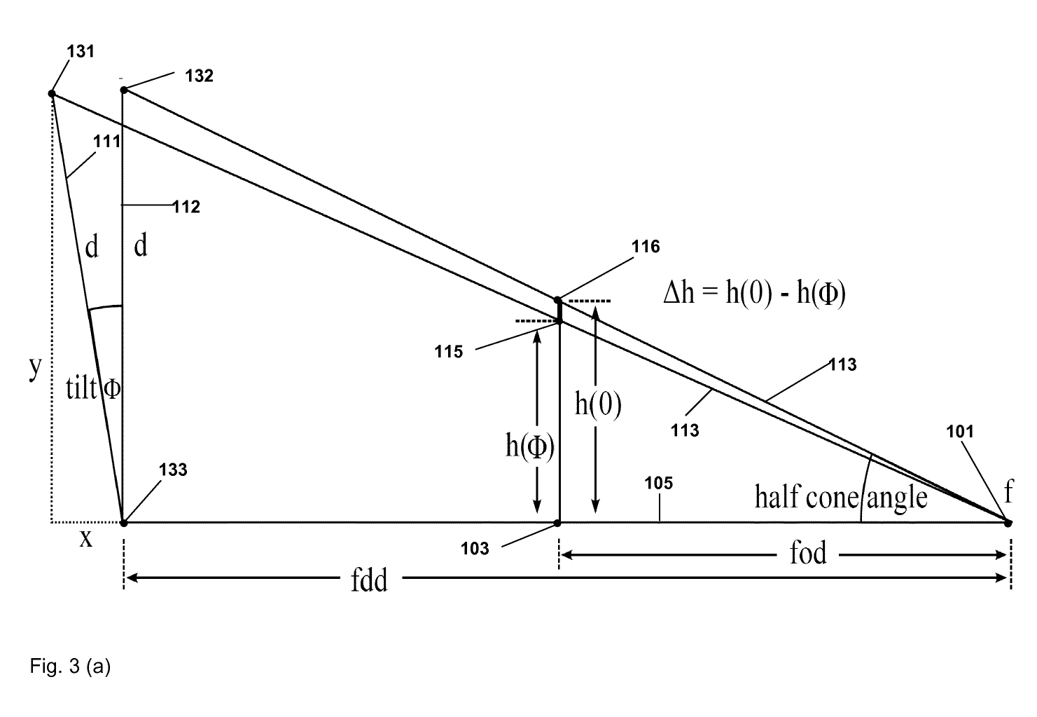

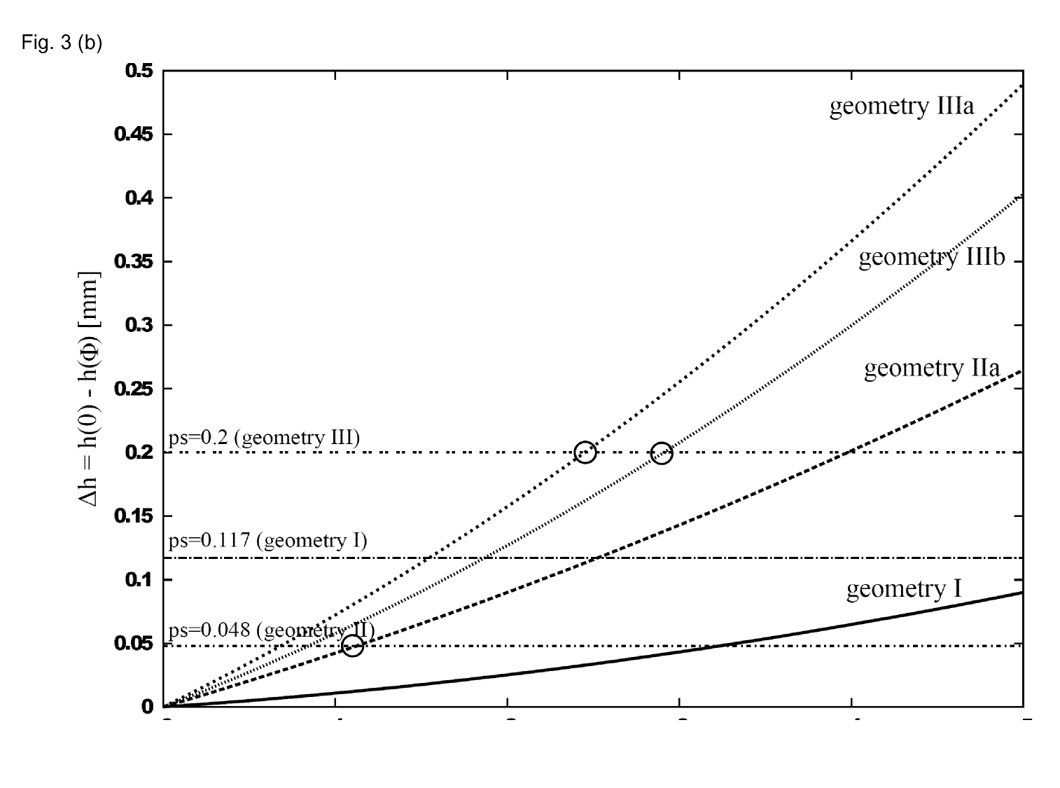

FIG. 3(a) illustrates effect of a deviation of the plane of the area 111 of the detector from the (ideal) x-y plane. While an ideal detector would have a x-ray sensitive area within the ideal plane 112 parallel to the x-y plane, a real detector has its area 111 or its x-ray sensitive area 111 tilted with respect to the ideal plane 112 by an angle phi. Thereby, the calibration object 115 is projected to a point 131 in the area 111 of the real detector which is a distance d away from the central point 133 of the real detector. Thereby, the calibration object 115 is arranged at a height which is smaller than a height of another calibration object 116, as illustrated in FIG. 3(a). Assuming that the detector lies in the ideal plane 112 this differently arranged calibration object 116 would be projected to a point 132 in the ideal detector plane 112 which has a same distance d from the central point 133 of the detector. Thus, when not taking into account the tilt of the real detector area 111 relative to the ideal detector area 112 the projection intensity peaks 132 would indicate a false positioning of the calibration object. Thus, taking into account the tilt of the real detector area relative to the ideal plane is necessary in order to accurately determine the geometry parameter values.

FIG. 3(b) illustrates further geometric relationships due to a tilt of the real detector area 111.

FIG. 3(a) introduces a function Δh(φ) to estimate the effect of an out-of-plane rotation error (tilt φ) to a point near the rotational axis, as a sample of a reconstruction volume which is centered around the rotational axis. Here, Δh(φ) is the offset of the intersection point of the rotational axis with a virtual ray which strikes the detector margin. FIG. 3(b) evaluates the maximum reconstruction error from 0 to 5 degrees. If we assume that the voxel spacing in the reconstruction is equal to or below the pixel spacing, the intersection of the error plot with the horizontal pixel spacing line indicates when the error exceeds one voxel. This can be evaluated for each geometry. Looking at geometry II this point is about 1°, for geometry III between 2-3° and within geometry I it is above 5°. This demonstrates that the influence of out-of-plane rotation errors depends to a great extent on the device geometry. Consequently, the out-of-plane angle precision of the calibration method must be evaluated with respect to this influence.

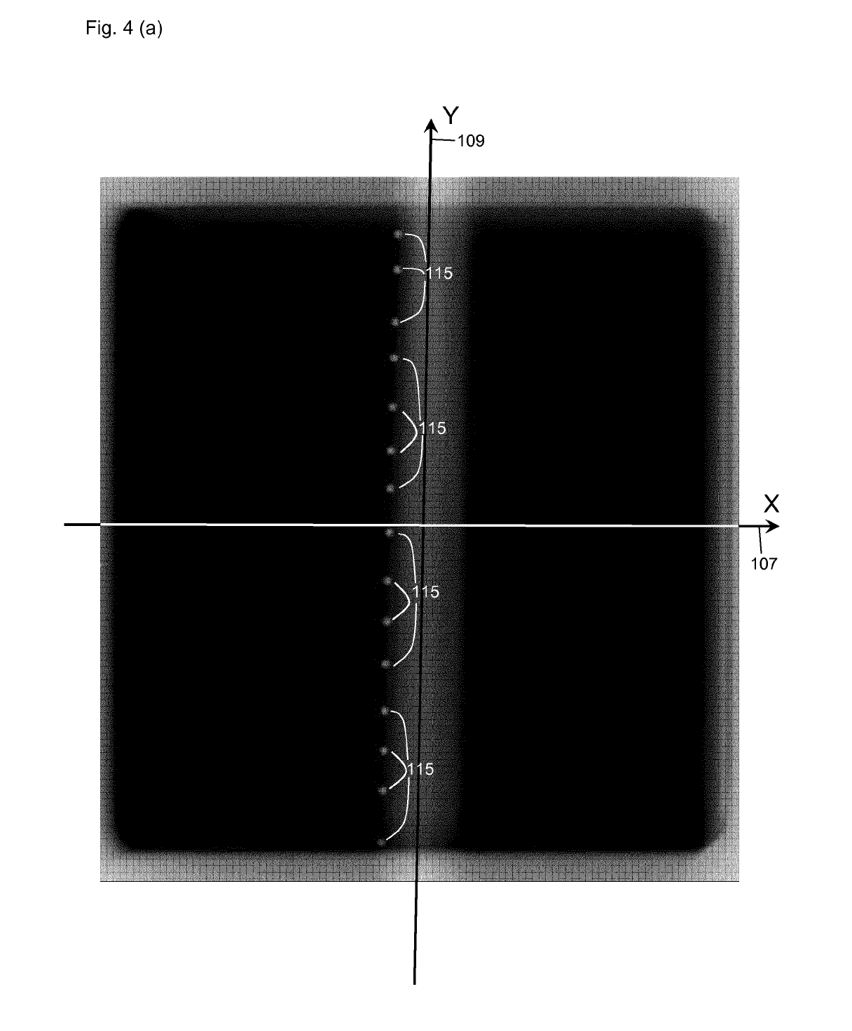

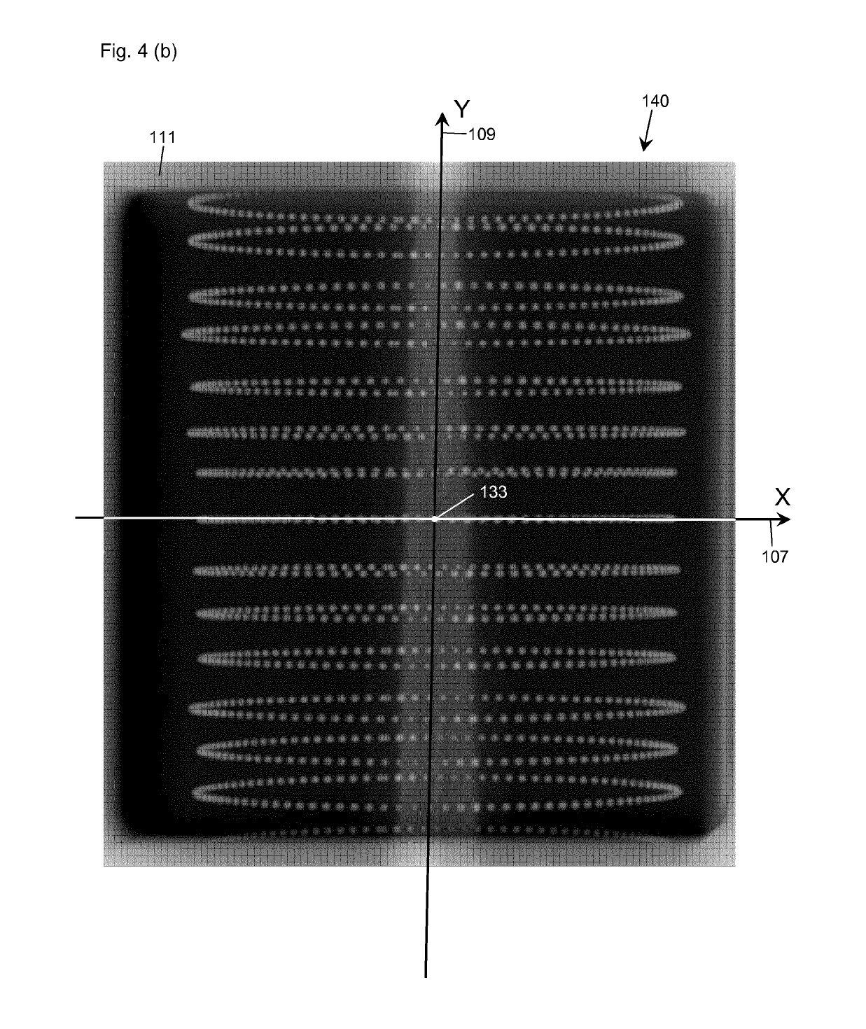

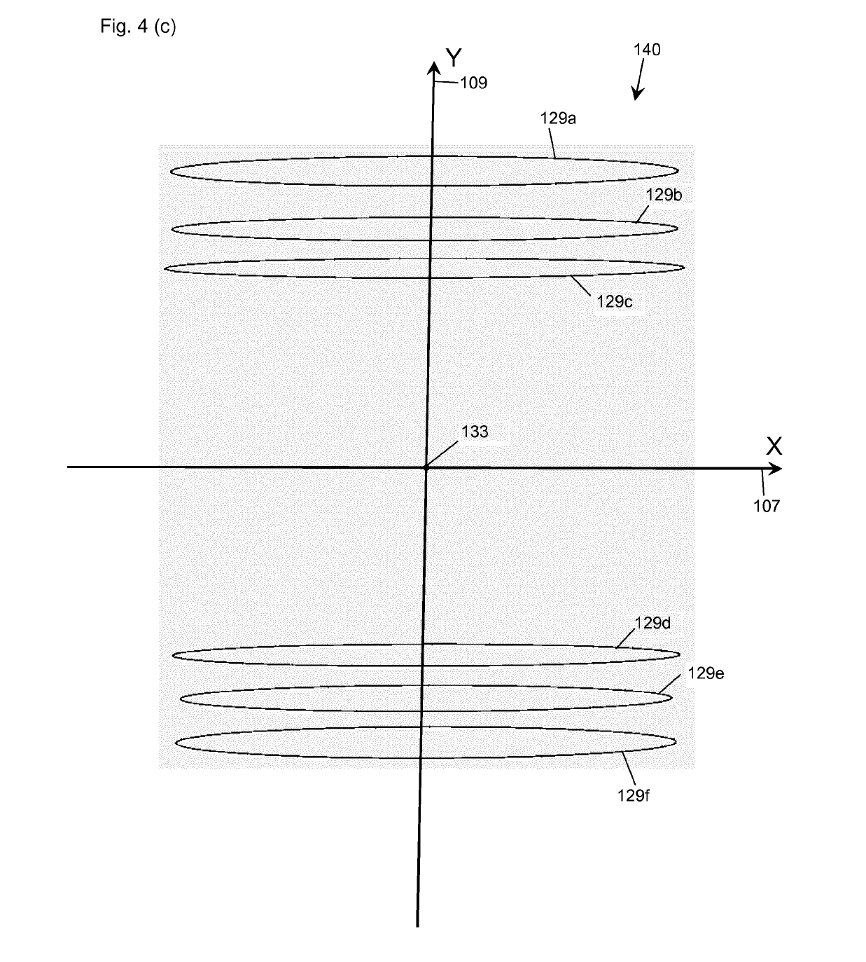

FIG. 4(a) schematically illustrates an arrangement of calibration objects for determining values of geometry parameters according to embodiment of the present invention. The calibration objects 115 are distributed along a line parallel to the rotation axis 109.

FIG. 4(b) schematically illustrates x-ray projection data 140 which are represented by individual intensity peaks forming ellipses 129 which are due to projections of the plural calibration objects 115 illustrated in FIG. 4(a) at different rotation angles.

As is apparent from FIG. 4(b), the ellipses 129 have about the same traverse diameter (an extent along the major axis, i.e. in the horizontal direction of FIG. 4(b)). However, the ellipses 129 have different conjugate diameters (extent along their minor axis, i.e. along the rotation axis 109, i.e. in the vertical direction of FIG. 4(b)). It is apparent, that the further the ellipses 129 are away from the central point 133 of the area 111 of the detector the larger is their conjugate diameter, i.e. their extent in the direction of the rotation axis 109. The further the ellipses are away from the central point 133 the better the ellipse orientations may be utilized for determining the values of the geometry parameters.

FIG. 4(c) illustrate other x-ray projection data 140 which may be utilized for determining values of geometry parameters of a cone beam computer tomography apparatus according to the embodiment of the present invention.

The x-ray projection data 140 comprise ellipse representations 129a, 129b, 129c, 129d, 129e, 129f which are due to projection of six different calibration objects arranged at different positions along a direction parallel to the rotation axis 109 but having about a same distance from the rotation axis 109. From x-ray projection data 140 captured on the area 111 of the detector the values of the geometry parameters can be determined according to embodiments of the present invention.

To calibrate the first mode (ƒod=800 mm) we used a phantom containing 17 metal ball bearings of 1 mm in diameter manually arranged more or less vertically on a wooden plate. In FIG. 4 one can see a projection image of this phantom along with an overlay of several images showing the ellipse trajectories generated by the rotation. From this we extracted six ellipses (FIG. 4(c)) as input for our optimization process without using a-priori information about the geometry of the calibration phantom. To extract the best possible ellipse information, the lower and upper three ellipses were used for the calibration.

### I. The Device Model

Our device model (see FIG. 1) is based on the following three assumptions: First, the X-ray source and the detector have to be statically coupled to one another, such that both have a static position and orientation in a common local coordinate frame. Often, this part of the device is called the C-arm of the CBCT. Second, we assume a flat-panel detector. Finally, the focus-detector-unit rotates about an arbitrary axis during image acquisition. Note that this axis 109 does not have to be aligned with the detector area 111 in any special way. These assumptions are nearly fulfilled for many dental C-arm CBCTs or Micro CTs.

The assumptions made above lead to a device model with fewer degrees of freedom compared to the general case, where each projection is calibrated individually. In fact, a rotational CBCT can be described by a set of eight parameters

q^{real}=[φ,σ,ψ,ƒod,ps,oₓ,o_{y},o_{z}] (1)

which are the distance of the focus to the rotational axis (ƒod), the pixel-size (ps), the position of the detector (oᵣ, o_{y}, o_{z}) and its orientation (φ,σ,ψ). Assuming a right-handed coordinate system in which the rotational axis corresponds to the y-axis and the focus is placed on the negative z-axis these eight parameters apply as follows. The focus f of the system is at f=(0,0,−ƒod)^{T}. Then, the local coordinate frame of the detector is given by a corner o=(oᵣ, o_{y}, o_{z})^{T} (the position of the detector) and two adjacent vectors

$$\begin{matrix}{d}^{x} = p sR ( \begin{matrix}1 \\ 0 \\ 0\end{matrix} ) \\ {d}^{y} = p sR ( \begin{matrix}0 \\ 1 \\ 0\end{matrix} ) \\ with & ( 2 ) \\ R = {R}_{x} ( φ ) {R}_{y} ( σ ) {R}_{z} ( ψ ) & ( 3 )\end{matrix}$$

which describes the orientation of the detector. Here, Rₓ(φ), R_{y}(σ), R_{z}(ψ) are rotation matrices about the x-, y- and z-axes, respectively. From equations (2) and (3) it follows that both vectors d^{x} and d^{y} are orthogonal to each other with the length of one pixel.

To introduce a projection matrix framework, the vectors d^{x}, d^{y}, o, f which represent the complete geometry information can be combined into the homogeneous calibration matrix \tilde{D}ε [symbol] ^{4×4} given by

$$\tilde{D} = [ \begin{matrix}{d}^{x} & {d}^{y} & f & o \\ 0 & 0 & 1 & 1\end{matrix} ] = ( \begin{matrix}{d}_{x}^{x} & {d}_{x}^{y} & 0 & {o}_{x} \\ {d}_{y}^{x} & {d}_{y}^{y} & 0 & {o}_{y} \\ {d}_{z}^{x} & {d}_{z}^{y} & - fod & {o}_{z} \\ 0 & 0 & 1 & 1\end{matrix} ) . \qquad ( 4 )$$

From \tilde{D} the projection matrix \tilde{P}ε [symbol] ^{3×4} can be derived, which projects a point in real-world coordinates onto the detector given by o, d^{x} and d^{y} and provides the point within the detector's local coordinate system. It is given as

$$\tilde{P} = \tilde{Z} {\tilde{D}}^{- 1} with \tilde{Z} = ( \begin{matrix}1 & 0 & 0 & 0 \\ 0 & 1 & 0 & 0 \\ 0 & 0 & 0 & 1\end{matrix} ) . \qquad ( 5 )$$

Here, \tilde{Z} is a simple orthogonal projection in z-direction.

During image acquisition the focus-detector-unit rotates about the y-axis 109 (see FIG. 1). If we assume that at a fixed time t, i.e. image number t, the device is rotated about the angle α(t) then the focus has the position (reference sign 101 in FIG. 1)

$${\tilde{R}}_{α ( t )} ( \begin{matrix}f \\ 1\end{matrix} )$$

and the detector unit is given by

$${\tilde{R}}_{α ( t )} ( \begin{matrix}o \\ 1\end{matrix} ) , {\tilde{R}}_{α ( t )} ( \begin{matrix}{d}^{x} \\ 1\end{matrix} ) and {\tilde{R}}_{α ( t )} ( \begin{matrix}{d}^{y} \\ 1\end{matrix} ) .$$

Thereby \tilde{R}_{α}ε [symbol] ^{4×4} performs a simple rotation about the y-axis through the angle α in a mathematically positive sense. As a consequence the detector matrix \tilde{D}ₜ, as well as the corresponding projection matrix \tilde{P}ₜ at time t are given by

\tilde{D}ₜ=\tilde{R}_{α(t)}\tilde{D}

and

\tilde{P}ₜ=\tilde{Z}\tilde{D}ₜ⁻¹=\tilde{Z}\tilde{D}⁻¹R_{α(t)}^{T}. (6)

Now the projection of a fixed point \tilde{x}ε [symbol] ⁴ at time t is given by

\tilde{p}ₜ=\tilde{P}ₜ\tilde{x}=\tilde{Z}\tilde{D}⁻¹\tilde{R}_{α(t)}^{T}\tilde{x}. (7)

### A. Normalizing the Geometry

From projection images themselves, the eight parameters of q^{real} defining the vectors d^{x}, d^{y}, o, f are only determinable with two ambiguities which leads to a system of equivalent geometry configurations with six degrees of freedom (DOF's). First we can freely choose the unit in which we measure the last five parameters of q^{real} as in equation (1). Thus, a change of the measurement unit corresponds to a uniform scaling

$$q = [ {q}_{1} , {q}_{2} , {q}_{3} , {q}_{4} , {q}_{5} , {q}_{6} , {q}_{7} , {q}_{8} ] {↦}_{{U}_{γ}} [ {q}_{1} , {q}_{2} , {q}_{3} , γ {q}_{4} , γ {q}_{5} , γ {q}_{6} , γ {q}_{7} , γ {q}_{8} ] . \qquad ( 8 )$$

While scaling the geometry of the system the spatial size of the reconstructed volume also gets scaled with the same factor γ. Vice versa, if one knows the real distance of two reconstructed points one can easily compute this scaling factor. Reconstructions provided by q and U_{γ}(q) are equal except for spatial scaling. Second, since we can only observe 2D-projections of 3D-points one can freely scale the size of the detector and its distance with respect to the position of the focus. More exactly this transformation S_{μ} looks like

$$q = [ {q}_{1} , {q}_{2} , {q}_{3} , {q}_{4} , {q}_{5} , {q}_{6} , {q}_{7} , {q}_{8} ] {↦}_{{S}_{μ} ∘ {U}_{γ}} [ φ , σ , ψ , 1 , \frac{2 p s}{{o}_{z} + fod} , \frac{2 {o}_{x}}{{o}_{z} + fod} , \frac{2 {o}_{y}}{{o}_{z} + fod} , 1 ] = {q}^{norm} . \qquad ( 9 )$$

Thereby the position of the focus and the size of volume remain fixed. Reconstructions provided by q and S_{μ}(q) are identical.

If we apply these transformations to the original geometry configuration q^{real} with

$$γ = \frac{1}{fod} and μ = \frac{2 fod}{{o}_{z} + fod}$$

we get a normalized version q^{norm} of the geometry:

$${q}^{real} = [ \begin{matrix}φ , σ , ψ , fod , \\ p s , {o}_{x} , {o}_{y} , {o}_{z}\end{matrix} ] {↦}_{{S}_{μ} ∘ {U}_{γ}} [ \begin{matrix}φ , σ , ψ , 1 , \frac{2 p s}{{o}_{z} + fod} , \\ \frac{2 {o}_{x}}{{o}_{z} + fod} , \frac{2 {o}_{y}}{{o}_{z} + fod} , 1\end{matrix} ] = {q}^{norm} . \qquad ( 10 )$$

Two parameters (q₄^{norm}, q₈^{norm}) of the normalized geometry q^{norm} are fixed to one. The remaining six entries, respectively ratios of the original geometry, can be determined just from the projection images without any a-priori information.

Now, if we form the vectors dₙₒᵣₘ^{x}, dₙₒᵣₘ^{y}, oₙₒᵣₘ, fₙₒᵣₘ defined by q^{norm} in analogy to the equations (1), (2) and (3) and combine these into a normalized calibration matrix \tilde{D}ₙₒᵣₘ it holds that

$$\begin{matrix}\begin{matrix}{\tilde{D}}_{norm} = [ \begin{matrix}{d}_{norm}^{x} & {d}_{norm}^{y} & {f}_{norm} & {o}_{norm} \\ 0 & 0 & 1 & 1\end{matrix} ] \\ = [ \begin{matrix}μ γ {d}^{x} & μ γ {d}^{y} & γ f & μ γ o + ( 1 - μ ) γ f \\ 0 & 0 & 1 & 1\end{matrix} ]\end{matrix} & ( 11 ) \\ = ( \begin{matrix}γ & 0 & 0 & 0 \\ 0 & γ & 0 & 0 \\ 0 & 0 & γ & 0 \\ 0 & 0 & 0 & 1\end{matrix} ) [ \begin{matrix}{d}^{x} & {d}^{y} & f & o \\ 0 & 0 & 1 & 1\end{matrix} ] ( \begin{matrix}μ & 0 & 0 & 0 \\ 0 & μ & 0 & 0 \\ 0 & 0 & 1 & ( 1 - μ ) \\ 0 & 0 & 0 & μ\end{matrix} ) & ( 12 ) \\ with γ = \frac{1}{fod} and μ = \frac{2 fod}{{o}_{z} + fod} . & \end{matrix}$$

For the normalized projection matrix \tilde{P}ₙₒᵣₘ it holds that

$$\begin{matrix}{\tilde{P}}_{norm} = \tilde{Z} {\tilde{D}}_{norm}^{- 1} = \tilde{Z} ( \begin{matrix}\frac{1}{μ} & 0 & 0 & 0 \\ 0 & \frac{1}{μ} & 0 & 0 \\ 0 & 0 & 1 & \frac{μ - 1}{μ} \\ 0 & 0 & 0 & \frac{1}{μ}\end{matrix} ) {\tilde{D}}^{- 1} ( \begin{matrix}\frac{1}{γ} & 0 & 0 & 0 \\ 0 & \frac{1}{γ} & 0 & 0 \\ 0 & 0 & \frac{1}{γ} & 0 \\ 0 & 0 & 0 & 1\end{matrix} ) & ( 13 ) \\ = \frac{1}{μ} \tilde{Z} {\tilde{D}}^{- 1} ( \begin{matrix}\frac{1}{γ} & 0 & 0 & 0 \\ 0 & \frac{1}{γ} & 0 & 0 \\ 0 & 0 & \frac{1}{γ} & 0 \\ 0 & 0 & 0 & 1\end{matrix} ) ≃ \tilde{Z} {\tilde{D}}^{- 1} ( \begin{matrix}\frac{1}{γ} & 0 & 0 & 0 \\ 0 & \frac{1}{γ} & 0 & 0 \\ 0 & 0 & \frac{1}{γ} & 0 \\ 0 & 0 & 0 & 1\end{matrix} ) & ( 14 )\end{matrix}$$

from which one can easily see that the projection matrices belonging to g^{real} and q^{norm} only differ in a uniform spatial scaling of the volume space. The symbol ≅ means projective equivalence.

To summarize, the normalized geometry q^{norm} has only six DOF's, while providing a reconstruction which only differs in a spatial scaling compared to a reconstruction based on the real geometry q^{real}. As demonstrated in this paragraph, it suffices to consider normalized CBCT geometries for both calibration and reconstruction without loss of generality. Hence, in the rest of this paper, we omit the superscript (•)^{norm} and assume a normalized geometry with a focus-to-object distance of ƒ od≡1 and a z-translation of o_{z}≡1 which correspond to the entries q₄ and q₈ of the geometry-defining parameter vector q (compare with equation (1)).

### II. MATHEMATICAL MODEL

In the following, we present all theoretical aspects of the calibration method.

### A. Main Concept

From equation (7) it is easy to see that the projection-curve of a fixed point in dependency of time t is identical to the projection of an orbit around the y-axis at time t=0. As a consequence we can drop the time component by considering circular orbits (labelled by 117 in FIG. 1) around the y-axis (called y-orbit in the following) instead of single points.

These y-orbits 117 get projected to ellipses 129. In the next section we will describe how to obtain these ellipses. Our approach is based on the fact that the ellipses 129 determined by the y-orbits 117 of the radio opaque markers 115 can be measured directly within the projections. This observation allows us to determine the unknown calibration matrix \tilde{D} and the y-orbits of the markers. In the following, we represent the ellipses as homogeneous symmetric matrices \tilde{C}ⁱε [symbol] ^{3×3}, i=1, . . . , n. An observed ellipse \tilde{C}ⁱ depends on the geometric configuration represented by the calibration matrix \tilde{D}, the radius rᵢ and the height hᵢ of the y-orbit of a marker.

A decomposition of the conic section equation describing the ellipses allows for a direct computation of the pair (rᵢ, hᵢ) when \tilde{C}ⁱ and \tilde{D} are given. More precisely, if one assumes a fixed calibration matrix \tilde{D} there is a bijection, mapping y-orbits defined by (rᵢ, hᵢ) onto observable ellipses \tilde{C}ⁱ in the image domain. We derive an explicit formula for this bijective mapping and much more important for its inversion. This explicit formula will be used to reduce the complexity (6 variables instead of 6+2n) of our optimization algorithm which determines the CBCT geometry.

In the next sections we prove that the resulting problem can be stated as follows: Given n ellipses \tilde{C}ⁱε [symbol] ^{3×3}, i=1, . . . , n measured from the projection images. Find a homography (i.e. a bijective projective mapping)

$$\tilde{G} = ( \begin{matrix}{d}_{x}^{x} & {d}_{x}^{y} & {o}_{x} \\ {d}_{y}^{x} & {d}_{y}^{y} & {o}_{y} \\ \frac{1}{2} {d}_{z}^{x} & \frac{1}{2} {d}_{z}^{y} & 1\end{matrix} ) \qquad ( 15 )$$

between the real detector plane and a canonical detector plane such that for some arbitrary (rᵢ, hᵢ)ε( [symbol] ⁺, [symbol] ) defining the canonical ellipses

$${\tilde{C}}_{c}^{i} = ( \begin{matrix}1 & 0 & 0 \\ 0 & \frac{1 - {r}_{i}^{2}}{{h}_{i}^{2}} & - \frac{2}{{h}_{i}} \\ 0 & - \frac{2}{{h}_{i}} & 4\end{matrix} ) \qquad ( 16 )$$

the following equation holds

\tilde{C}ⁱ≅\tilde{G}^{T}\tilde{C}_{c}ⁱ\tilde{G}=1, . . . , n. (17)

This simple algebraic representation of the relationship of CBCT-geometry, y-orbit and observed ellipse can be achieved by temporarily adding a canonical detector plane (given by the matrix \tilde{D}_{c}, see section II C) and afterwards mapping the canonical detector to the real detector (see FIG. 2). As mentioned previously equation (17) can be solved explicitly for (rᵢ, hᵢ). The matrix G contains the complete geometric information of the CBCT required for reconstruction.
B. Identifying the Trajectory of a Fixed Point with a Conic Section

Let \tilde{x}ε [symbol] ⁴ be a homogeneous representation of a point on an orbit with radius r and height h. Then \tilde{x} has the representation

\tilde{x}=(rx₁,h,rx₂,1) (18)

with x₁²+x₂²=1. Now define \tilde{W}ε [symbol] ^{4×3} and \tilde{y}ε [symbol] ³ with

$$\tilde{W} = ( \begin{matrix}r & 0 & 0 \\ 0 & 0 & h \\ 0 & r & 0 \\ 0 & 0 & 1\end{matrix} ) \\ and \\ \tilde{y} = ( \begin{matrix}{x}_{1} \\ {x}_{2} \\ 1\end{matrix} ) . \qquad ( 19 )$$

That means

\tilde{x}=\tilde{W}\tilde{y} (20)

with \tilde{y} being a point on the two-dimensional unit circle. The projection \tilde{P} maps the point \tilde{x} to image coordinates

\tilde{p}=\tilde{P}\tilde{x}=\tilde{P}\tilde{W}\tilde{y}. (21)

Note that both \tilde{P}ε [symbol] ^{3×4} and \tilde{W}ε [symbol] ^{4×3} are not square matrices, in contrast to \tilde{P}\tilde{W} which is square and invertible (except for unrealistic detector geometries).

Since \tilde{y} is a point on a unit circle the following conic section equation holds:

$$0 = {\tilde{y}}^{T} \tilde{K} \tilde{y} \\ with \\ \tilde{K} = ( \begin{matrix}1 & 0 & 0 \\ 0 & 1 & 0 \\ 0 & 0 & - 1\end{matrix} ) . \qquad ( 22 )$$

With \tilde{y}=(\tilde{P}\tilde{W})⁻¹\tilde{p} we derive the following equation for the image point \tilde{p}

0=\tilde{y}^{T}\tilde{K}\tilde{y}=\tilde{p}^{T}\tilde{C}\tilde{p} (23)

where

\tilde{C}=(\tilde{P}\tilde{W})^{-T}\tilde{K}(\tilde{P}\tilde{W})⁻¹. (24)

In summary, this means that, through the perspective projection, the orbit 117 with radius r (119) and height h (121) around the y-axis will be mapped to the conic section \tilde{C}. In our case these conic sections are ellipses. Furthermore with (5) we find:

\tilde{C}=(\tilde{Z}\tilde{D}⁻¹\tilde{W})^{-T}\tilde{K}(\tilde{Z}\tilde{D}⁻¹\tilde{W})⁻¹ (25)

with non-square matrices \tilde{Z}ε [symbol] ^{3×4}, \tilde{W}ε [symbol] ^{4×3} and square matrices \tilde{D}ε^{4×4}, \tilde{K}ε [symbol] ^{3×3}.

### C. Decomposition of the Conic Section Equation

Let's define a canonical calibration matrix \tilde{D}_{c}ε [symbol] ^{4×4} with

$${\tilde{D}}_{c} = ( \begin{matrix}1 & 0 & 0 & 0 \\ 0 & 1 & 0 & 0 \\ 0 & 0 & - 1 & 1 \\ 0 & 0 & 1 & 1\end{matrix} ) \qquad ( 26 )$$

and a projective mapping \tilde{G}′ε [symbol] ^{4×4} with

$${\tilde{G}}^{′} = ( \begin{matrix}{d}_{x}^{x} & {d}_{x}^{y} & 0 & {o}_{x} \\ {d}_{y}^{x} & {d}_{y}^{y} & 0 & {o}_{y} \\ - \frac{1}{2} {d}_{z}^{x} & - \frac{1}{2} {d}_{z}^{y} & 1 & 0 \\ \frac{1}{2} {d}_{z}^{x} & \frac{1}{2} {d}_{z}^{y} & 0 & 1\end{matrix} ) . \qquad ( 27 )$$

Then \tilde{D}=\tilde{D}_{c}\tilde{G}′ and the conic section equation (25) can be decomposed into

\tilde{C}=(\tilde{Z}\tilde{G}′⁻¹\tilde{D}_{c}⁻¹\tilde{W})^{-T}\tilde{K}(\tilde{Z}\tilde{G}′⁻¹\tilde{D}_{c}⁻¹\tilde{W})⁻¹. (28)

Using (15) and the fact that \tilde{Z}\tilde{G}′⁻¹=G⁻¹\tilde{Z} we get

\tilde{C}=(\tilde{G}⁻¹\tilde{Z}\tilde{D}_{c}⁻¹\tilde{W})^{-T}\tilde{K}(\tilde{G}⁻¹\tilde{Z}\tilde{D}_{c}⁻¹\tilde{W})⁻¹. (29)

Since G is square and invertible it can be factored out and it follows that

\tilde{C}=\tilde{G}^{T}(\tilde{Z}\tilde{D}_{c}⁻¹\tilde{W})^{-T}\tilde{K}(\tilde{Z}\tilde{D}_{c}⁻¹\tilde{W})⁻¹\tilde{G}. (30)

With

\tilde{C}_{c}=(\tilde{Z}\tilde{D}_{c}⁻¹\tilde{W})^{-T}\tilde{K}(\tilde{Z}\tilde{D}_{c}⁻¹\tilde{W})⁻¹ (31)

equation (30) simplifies to

\tilde{C}=\tilde{G}^{T}\tilde{C}_{c}\tilde{G}. 32)

Note that \tilde{C}_{c} only depends on the radius and the height of the orbit. In particular it is independent of the device geometry. Substituting (5), (19), (22) and (26) into equation (31) we get the explicit representation

$${\tilde{C}}_{c} = \frac{1}{{r}^{2}} ( \begin{matrix}1 & 0 & 0 \\ 0 & \frac{1 - {r}^{2}}{{h}^{2}} & - \frac{2}{h} \\ 0 & - \frac{2}{h} & 4\end{matrix} ) ≃ ( \begin{matrix}1 & 0 & 0 \\ 0 & \frac{1 - {r}^{2}}{{h}^{2}} & - \frac{2}{h} \\ 0 & - \frac{2}{h} & 4\end{matrix} ) \qquad ( 33 )$$

of the canonical conic section defined by a y-orbit with hight h and radius r.

### III. Optimization Process

Before solving the conic section equation (17) in the optimization process, we have to consider some preprocessing steps to obtain the elliptic projection trajectories \tilde{C}ⁱ in the given X-ray images. In our approach any kind of point-like, radio-opaque markers can be used. For all recovering processing steps standard methods in imaging science exist, as is know by the skilled person. We extract the midpoints of our metal ball bearings using a border segmentation followed by an elliptical Hough transformation, both with sub-pixel precision. Subsequently or along the way, each trajectory can be tracked e.g. by a Kalman filter and optical flow procedures. To fit ellipses to the point set a method similar to the standard approach by Fitzgibbon et al.² may be used.

Given n ellipses \tilde{C}ⁱ one has to find a normalized geometry vector q^{norm} (defining the homography \tilde{G}) such that (17) holds for some arbitrary (rᵢ, hᵢ)ε( [symbol] ⁺, [symbol] ) defining \tilde{C}_{c}ⁱ as in equation (16). The implemented optimization process is illustrated as algorithm 1 below.

Algorithm 1 is a simple random search in the geometry vector q^{norm} (6 DOF) combined with an annealing process which minimizes an objective function \hat{ƒ} that will be described in the next paragraph. The annealing process itself is implemented by shrinking (by a factor

Algorithm 1: Local optimization process

Input: ellipses \tilde{C}¹, . . . , \tilde{C}ⁿ, search window qₘᵢₙ ≦ qₘₐₓ
K Number of iterations, J Number of shrinkage steps, I Number of random
samples, δ shrinkage factor
Output: calibration vector q^{opt} (local optimum)
${q}^{opt} = \frac{{q}_{min} + {q}_{max}}{2}$
for k ← 1, . . . , K do

| \| | qₘᵢₙ^{c} = qₘᵢₙ | | |
|---|---|---|---|
| \| | qₘₐₓ^{c} = qₘₐₓ | | |
| \| | for j ← 1, . . . , J do | | |
| \| | \| | // Random samples | |
| \| | \| | for i ← 1, . . . , I do | |
| \| | \| | \| | q^{r} = randomVector (qₘᵢₙ^{c}, qₘₐₓ^{c}) |
| \| \| | \| \| | \| \| | ${q}^{opt} = {arg min}_{x ∈ \{ {q}^{opt} , {q}^{r} \}} \hat{f} ( x , {\tilde{C}}^{1} , … , {\tilde{C}}^{n} )$ |
| \| | \| | end | |
| \| | \| | // Shrinkage | |
| \| | \| | w = qₘₐₓ^{c} − qₘᵢₙ^{c} | |
| \| | \| | ${q}_{min}^{c} = {q}^{opt} - δ \frac{w}{2}$ | |
| \| | \| | ${q}_{max}^{c} = {q}^{opt} + δ \frac{w}{2}$ | |
| \| | \| | // Adjusting the search window if needed | |
| \| | \| | if qₘᵢₙ^{c} < qₘᵢₙ then | |
| \| | \| | \| | qₘₐₓ^{c} = qₘₐₓ^{c} + (qₘᵢₙ − qₘᵢₙ^{c}) |
| \| | \| | \| | qₘᵢₙ^{c} = qₘᵢₙ |
| \| | \| | end | |
| \| | \| | if qₘₐₓ^{c} > qₘₐₓ then | |
| \| | \| | \| | qₘᵢₙ^{c} = qₘᵢₙ^{c} − (qₘₐₓ^{c} − qₘₐₓ) |
| \| | \| | \| | qₘₐₓ^{c} = qₘₐₓ |

| | | |
|---|---|---|
| \| | \| | end |

| | |
|---|---|
| \| | end |

end
return q^{opt}

0<δ<1, J times) a box-like search window centered around the current optimum q^{opt} after a fixed number I of random samples within this box. To cope with local minima we restart the search K times. The local optimization process is illustrated in detail in algorithm 1.

The box-like search window qₘᵢₙ≦q≦qₘₐₓ (pointwise) is defined by six degrees of freedom of the normalized geometry vector q^{norm}. Throughout this paper, we use the same initial conditions for all calibrations of simulated as well as real data sets. These are

K=100, J=1000, I=10, δ=0.99, (34)

qₘᵢₙ=(−10°,10°,10°,1,2×10⁻⁴,3000×10⁻⁴,3000×10⁻⁴,1), (35)

qₘₐₓ=(+10°,+10°,+10°,1,2×10⁻³,0,0,1). (36)

The search window qₘᵢₙ≦q≦qₘₐₓ is chosen such that it covers a large class of real CBCT devices. This includes detector rotations up to ±10°, a focus-to-detector distance between 10³ and 10⁴ pixel

$$( \frac{{q}_{4} + {q}_{8}}{{q}_{5}} )$$

as well as detector translations between −1500 and 0 pixel

$$( \frac{{q}_{6}}{{q}_{5}} and \frac{{q}_{7}}{{q}_{5}} ) ,$$

independent from the absolute dimensions of the device. Given a pixel spacing of 0.05 mm, as a micro CT example, qₘᵢₙ and qₘₐₓ admits of feasible focus-to-detector distances ranging from 50 mm to 500 mm.

The global objective function \hat{ƒ} is the sum of individual objective functions

$$\begin{matrix}\hat{f} ( q , {\tilde{C}}^{1} , … , {\tilde{C}}^{n} ) = {\sum }_{i = 0}^{n} f ( q , {\tilde{C}}^{i} ) \\ with & ( 37 ) \\ f ( q , {\tilde{C}}^{i} ) = match ( {\tilde{C}}^{i} , {\tilde{G}}_{q}^{T} correct ( {\tilde{G}}_{q}^{- T} {\tilde{C}}^{i} {\tilde{G}}_{q}^{- 1} ) {\tilde{G}}_{q} ) & ( 38 )\end{matrix}$$

being the objective function for a single ellipse. The matrix \tilde{G}_{q}ε [symbol] ^{3×3} is uniquely defined by the geometry vector q in analogy to equations (1),(2),(3) and (15). Note that the matrix \tilde{G}_{q} is invertible iff. the focus does not lie in the detector plane. This will be guaranteed by the initial search window. Thereby the match( ) function is a heuristic that quantifies the match of two ellipses. It is implemented as the sum of the absolute differences of four corresponding points which are given as the intersections of the ellipse curve with both principal axes. These four points can be determined from the defining conic section matrix \tilde{C} by simple algebra. The function correct (Ĉ) normalizes the matrix Ĉ by division with its top-left entry and forces the structure

$$( \begin{matrix}1 & 0 & 0 \\ 0 & * & * \\ 0 & * & 4\end{matrix} ) . \qquad ( 39 )$$

Taking this into account, the term \tilde{G}_{q}^{T} correct (\tilde{G}_{q}^{-T}\tilde{C}\tilde{G}_{q}⁻¹)\tilde{G} in equation (38) is an approximation for the projection of the y-orbit (r, h) which matches the ellipse \tilde{C} best (for a fixed candidate \tilde{G}_{q}). As a consequence of the invertibility of \tilde{G}_{q} the matched ellipse pairs are non-degenerated iff. the input ellipses are non-degenerated. The real best-fitting y-orbit is given by

$${arg min}_{( r , h )} match ( \tilde{C} , {\tilde{G}}_{q}^{T} ( \begin{matrix}1 & 0 & 0 \\ 0 & \frac{1 - {r}^{2}}{{h}^{2}} & - \frac{2}{h} \\ 0 & - \frac{2}{h} & 4\end{matrix} ) {\tilde{G}}_{q} ) . \qquad ( 40 )$$

From (17), it follows that for the correct \tilde{G}_{q} the conic section \tilde{G}_{q}^{-T}\tilde{C}\tilde{G}_{q}⁻¹ (after normalization) is of the form (39). Consequently we can do the correction step (and so the approximation) by forcing the known components of \tilde{G}_{q}^{-T}\tilde{C}\tilde{G}_{q}⁻¹ to the correct values (see also equations (32) and (33) in section II C). Note that this approximation step reduces the number of variables in the optimization process dramatically. The approximated objective function acts as an upper bound to the desired objective function but with the same optimum and the same optimal value in an error-free setting. In such a case the objective function \hat{ƒ}(q, \tilde{C}¹, . . . , \tilde{C}ⁿ) is zero for the correct geometry q, otherwise it is greater than zero. Nevertheless, in the case of corrupted input ellipses \tilde{C}ⁱ, there is no proof that the match(·) function is optimal in the sense that the approximated objective function (Eq. (38)) and the desired one (Eq. (40)) share the optimum.

Geometric calibration refers to knowing the exact scan geometry of the acquisition geometry with very high precision. Geometric accuracy is fundamental in image reconstruction to avoid typical misalignment errors. Visible artifacts occur even from minute deviation of the true geometry from the desired geometry. On the other hand, it is well known that mechanical stability, which permits off-line calibration and repeatability, is very important for typical C-arm-based systems such as CBCTs since the systems are not as stable as gantry-based CTs. Hence it is obvious that simple calibration procedures are a very important prerequisite to obtain high-quality 3D reconstructions.

It is introduced here a novel calibration method for CBCT flat-panel machines with a circular image acquisition orbit (360°). It is an off-line procedure, i.e. the geometric parameters are determined in a scan before the system is operated on patients. This assumption seems reasonable for sufficiently stable machines. Our method is based on a simple phantom, that does not require accurate fabrication with only minute tolerances. Any point-like highly-dense objects can be used. However, the tiny point-like radio-opaque markers in our phantom should be distributed such that they produce as large as possible ellipses on the detector over the circular orbit. In order to obtain accurate calibration results, it may be desirable to position the markers in such a way that their projection orbits extend over the full detector width Already, a more or less vertical line of markers which is positioned some distance away from the rotation axis (which in many devices is indicated by a laser beam crossing) fulfills this requirement. Such a phantom may easily be produced manually within a few minutes, e.g. by placing metal balls taken out of a ballpoint pen in a wax-plate or acrylic plate. If the resulting ellipses do not fulfill the conditions explained above, the objects can easily be replaced in other positions until the observed ellipses are large enough and have sufficiently long minor axes. Such a phantom is very affordable to produce and also very flexible. Calibration can be performed time-efficiently in 1-5 minutes on an up-to-date laptop computer. Thus, if fully implemented in software, it could be used for repeated recalibration by the user. From the ellipses, seven parameters that completely describe a CBCT scanner with truly circular acquisition geometry can be determined. By combining two parameters into a ratio and normalizing this ratio, these seven parameters are reduced to six. This step is a fundamental prerequisite for our mathematical solution to determine the unknown calibration matrix \tilde{D}ₜ from the observed ellipses \tilde{C}ⁱ. A significant contribution of this application may be the decomposition of the conic section equations of the ellipses. From our empirical observations it is observed that it may be better to have few (>4) clearly defined ellipses rather than many ellipses which also include some degenerated ones.

The presented approach is capable of calibrating detector tilt and slant, i.e. the two out-of-plane angles φ and σ. Theoretical results herein described demonstrate that the larger the cone angle, the larger is the effect of the out-of-plane angles. The cone angles of the machines to be calibrated may range between 4.9 and 14 degrees. Regardless of the overall effect of these two out-of-plane errors on the reconstruction, theoretical results derived herein indicate, that the method introduced here is capable of substantially reducing the error. An important finding is, that the proposed method is capable of calibrating the out-of-plane angles with increasing accuracy in cases where their effect also increases. In other words, for larger cone angles when neglecting out-of-plane angles negatively affects reconstruction accuracy, our method becomes more effective and accurate.

It may be advisable to define a reasonable search bounding box based on manufacturer specifications and approximate estimations. Using the described methods of embodiments enable to calibrate devices such as a micro CBCT as well as a dental CBCT with the same initial conditions of the optimization process. All parameters may be determined in units u, i.e. the focus-to-detector distance. Scaling could be determined either by knowledge of the true distance of details in an object or by knowing e.g. the focus-to-detector distance plus pixel size. An advantage may be that fabrication errors in the phantom cannot propagate into calibration errors. The unknown distribution of the point-markers in our phantom (i.e. calibration objects) makes it impossible to provide information on angular spacing between the projections. Therefore, the angle may be estimated by dividing the rotation angle (2π in our cases) by the number of projections. This simple estimation is based on the assumption of a rather uniform circular movement of the source-detector unit. Scale-invariant calibration suitable for a large class of CBCTs and low restrictions on initial conditions of the optimization process are the major reasons which qualify the method according to embodiments of the present invention for being a starting point for more complex calibration procedures, e.g. when each image is calibrated separately.

It should be noted that the term comprising does not exclude other elements or steps and a or an does not exclude a plurality. Also elements described in association with different embodiments may be combined. It should also be noted that reference signs in the claims should not be construed as limiting the scope of the claims.

### REFERENCES

¹Yang, K., Kwan, A. L. C., Miller, D. F., Boone, J. M.: A geometric calibration method for cone beam ct systems. Med Phys 33(6)(6), 1695-1706 (2006)
²Pilu, M., Fitzgibbon, A., Fisher, R.: Ellipse-specific direct least-square fitting. In: Proc. International Conference on Image Processing. vol. 3, pp. 599-602 (1996)

1. Method for determining values of geometry parameters of a cone beam computer tomography apparatus, the geometry parameters in particular specifying an arrangement of a two-dimensional x-ray detector, an arrangement of a rotation axis of a relative rotation between an object and a mechanically coupled x-ray source/detector system, and an arrangement of a focus of the x-ray source, the method comprising:
obtaining x-ray projection data captured by the detector from at least three calibration objects arranged at mutually different positions, the x-ray projection data comprising for each calibration object plural projections at different rotation angles;
determining for each calibration object a respective ellipse representation from the respective plural projections;
performing a random search across candidate values of the geometry parameters for determining the values of the geometry parameters, wherein a cost function depending on the ellipse representations and the geometry parameters is optimized.

2. Method according to claim 1, wherein the performing the random search comprises:
describing each ellipse representation in the form of a virtual ellipse in a plane parallel to the rotation axis modified by a geometry related mapping defined by the geometry parameters.

3. Method according to claim 2, wherein describing the ellipse representation for each calibration object comprises the relation:
\check{C}=\tilde{G}^{T}·\check{C}c·\tilde{G},
wherein
\check{C} is the ellipse representation as determined from the projections of the calibration object,
\check{C}c is the virtual ellipse in the plane parallel to the rotation axis, and
\tilde{G} is the geometry related mapping depending on the geometry parameters, \tilde{G}^{T} denoting the transpose of \tilde{G},
wherein \check{C}, \check{C}c, and \tilde{G} are representable as 3×3 matrices.

4. Method according to claim 3, wherein performing the random search comprises:
finding the values of the geometry parameter such that the relation is at least approximately satisfied for all ellipse representations of the calibration objects by minimization of the cost function.

5. Method according to claim 2, wherein the cost function comprises a sum of individual cost functions, wherein each individual cost function is associated with a respective ellipse representation of one of the calibration objects and measures a deviation between the ellipse representation (\check{C}) and the virtual ellipse modified by the geometry related mapping, wherein the deviation is based on a sum of absolute differences of four points of the ellipse representation and the modified virtual ellipse, respectively, the four points being in particular defined as intersections of the principal axes of the ellipse with the respective ellipse.

6. Method according to claim 2, wherein the virtual ellipse exclusively depends on a position (h) along the rotation axis (y) of the respective calibration object and a distance (r) from the rotation axis (y) of the respective calibration object.

7. Method according to claim 1, wherein performing the random search in the geometry parameters comprises establishing start search ranges in the geometry parameters, which are explored during the random search, wherein the start search ranges are universal for different cone beam computer tomography apparatuses,
wherein in particular for each geometry parameter a start search range by a lower bound and an upper bound is specified.

8. Method according to claim 1, further comprising, after performing the random search in the geometry parameters to obtain preliminary values of the geometry parameters:
performing an annealing process further minimizing the cost function,
wherein the annealing process includes establishing annealing search ranges around the preliminary values of the geometry parameters, wherein the annealing search ranges include a narrower range of values of the geometry parameters than the start search ranges.

9. Method according to claim 8, wherein plural annealing processes are successively performed, in which sizes of the annealing search ranges are gradually decreased after a fixed number of random searches have been performed, wherein the values of the geometry parameters are determined as a minimum of the cost function over all performed random searches using search ranges having different sizes.

10. Method according to claim 1, wherein the geometry parameters represent normalized geometry parameters from which real geometry parameters can be calculated by spatial scaling.

11. Method according to claim 10, wherein the geometry parameters include information indicative of the six quantities:
(phi, sigma, psi) being three Euler angles describing the orientation of a x-ray sensitive area of the detector;
ps/fdd;
ox/fdd; and
oy/fdd, wherein
ps is the pixel size of a pixel of the x-ray sensitive area of the detector;
fdd is the distance along the z-axis between a focus of the x-ray source and the detector;
ox is an offset along the x-axis of an origin of the x-ray sensitive area of the detector and an x-coordinate of the focus of the x-ray source;
oy is an offset along the y-axis of an origin of the x-ray sensitive area of the detector and an y-coordinate of the focus of the x-ray source,
wherein the y-axis represents the rotation axis,
wherein the z-axis represents an axis perpendicular to the rotation axis through the focus of the x-ray source, and
wherein the x-axis represents an axis perpendicular to the y-axis and to the z-axis.

12. Method according to claim 1, wherein the area of the detector is flat and is oriented traverse to x-y-plane, wherein
phi indicates an rotation angle around the x-axis,
sigma indicates an rotation angle around the z-axis, and
psi indicates a rotation angle around the y-axis, to describe the orientation a x-ray sensitive area of the detector relative to an area of an ideal detector being oriented parallel to, in particular within, the x-y-plane,
wherein in particular
fod is the distance along the negative z-axis between the focus of the x-ray source and the rotation axis, and
oz is the distance along the z-axis between the detector and the rotation axis such that fdd=fod+oz.

13. Method according to claim 1, wherein each of the calibration objects comprises x-ray absorbing material distributed such as to result in an intensity distribution in a x-ray projection having a single peak such that a position of a center of absorption is derivable from the respective projection,
wherein in particular each calibration object comprises a metal sphere, in particular having a diameter between 0.5 mm and 2 mm.

14. Method according to claim 1, wherein relative positioning of the calibration objects is not used and/or not known to determine the values of the geometry parameters.

15. Method according to claim 1, wherein for each calibration object the respective plural projections are obtained at different rotating angles covering a range from 0 to 360, wherein a number of projections for each calibration object is between 50 and 200.

16. Method according to claim 1, wherein each calibration object is arranged such that for all rotation angles the calibration object is projected onto the x-ray sensitive area of the detector.

17. Method according to claim 1, wherein each calibration object has a distance (r) from the rotation axis such that intensity peaks within the plural projections of a calibration object due to the projection of the calibration object have a maximal mutual distance along the x-axis which is between 70% and 100% of an extent of the detector along the x-axis, the x-axis representing an axis perpendicular to the rotation axis and perpendicular to an axis which is perpendicular to the rotation axis and runs through the focus of the x-ray source.

18. Method according to claim 1, wherein the calibration objects are distributed (h) in a direction parallel to the rotation axis such that between 50% and 100% of the intensity in the x-ray projection data are captured in a first region and a second region of the x-ray sensitive area of the detector,
the first region and second region:
each having an extent of 30% of an extent of the area of the detector in a direction parallel to the rotation axis and
each including a respective outer edge of the area of the detector in the direction parallel to the rotation axis.

19. Method according to claim 1, wherein the calibration objects are positioned and/or oriented such that the plural projections of one of the calibration objects mutually do not overlap with the plural projections of another one of the calibration objects.

20. Method according to claim 1, wherein the calibration objects include between three and eight calibration objects which are distributed within a reconstruction volume, in particular located at a surface of a circular cylinder, in particular along a straight line parallel to the rotation axis.

21. Method according to claim 1, wherein
determining for each calibration object a respective ellipse representation from the respective plural projections comprises at least one of the following:
combining all projections of one calibration object into a single image;
extracting centers of the calibration object for all projections of the calibration object;
applying a border segmentation;
performing an elliptical Hough transformation;
applying a Kalman filter; and
fitting of an ellipse to all extracted and/or processed centers.

22. Method for deriving values of geometry parameters of a cone beam computer tomography apparatus, the method comprising:
performing an x-ray measurement to capture x-ray projection data of calibration objects;
determining, based on the x-ray projection data of the calibration objects, the values of the geometry parameters of the cone beam computer tomography apparatus according to claim 1.

23. Method of operating a cone beam computer tomography apparatus, the method comprising:
performing a method according to claim 1;
performing a further x-ray measurement to capture further x-ray projection data of an examination object different from the calibration objects;
using the values of geometry parameters to reconstruct a volume density of the examination object based on the further x-ray projection data.

24. Program element, which, when being executed by a processor, is adapted to control or carry out a method according to claim 1.

25. Computer-readable medium, in which a computer program is stored which, when being executed by a processor, is adapted to control or carry out a method according to claim 1.

26. Arrangement for determining values of geometry parameters of a cone beam computer tomography apparatus, the geometry parameters in particular specifying an arrangement of a two-dimensional x-ray detector, an arrangement of a rotation axis of a relative rotation between an object and a mechanically coupled x-ray source/detector system, and an arrangement of a focus of the x-ray source, the arrangement comprising:
an input section adapted to obtain x-ray projection data captured by the detector from at least three calibration objects arranged at mutually different positions, the x-ray projection data comprising for each calibration object plural projections at different rotation angles; and
a processor adapted:
to determine for each calibration object a respective ellipse representation from the respective plural projections, and
to perform a random search across candidate values of the geometry parameters for determining the values of the geometry parameters, wherein a cost function depending on the ellipse representations and the candidate values is optimized.

27. Cone beam computer tomography apparatus, comprising:
a two-dimensional x-ray sensitive detector;
a x-ray source adapted to generate x-rays originating from a focus and mechanically coupled to the detector; an object holder for holding an object; and
an arrangement according to claim 26,
wherein the apparatus is adapted to allow a relative rotation between the object holder and the mechanically coupled x-ray source/detector system.
